(19) United States
(12) Patent Application Publication
(10) Pub. No.: US 2015/0338423 A1
(43) Pub. Date: Nov. 26, 2015

(54) METHODS FOR DETERMINING RELATIVE BINDING ENERGY OF MONOMERS AND METHODS OF USING THE SAME

(71) Applicant: SABIC Global Technologies B.V., Bergen op Zoom (NL)

(72) Inventors: Sharankumar G. Shetty, Bangalore (IN); Jan Henk Kamps, Bergen op Zoom (NL); Girish Chandra, Bangalore (IN); Akhilesh Tanwar, Bangalore (IN); Robert R. Gallucci, Mt. Vernon, IN (US); Venkata Ramanarayanan Ganapathy Bhotla, Bangalore (IN); Edward J. Nesakumar, Bangalore (IN); Minor Senthil Kumar, Bangalore (IN)

(73) Assignee: SABIC Global Technologies B.V., Bergen op Zoom (NL)

(21) Appl. No.: 14/284,040

(22) Filed: May 21, 2014

(51) Int. Cl. G01N 33/74; G06F 19/18; C08G 64/20

(52) U.S. Cl. CPC G01N 33/743; C08G 64/20; G06F 19/18; G01N 2333/70567

(57) ABSTRACT

Disclosed herein are monomers that exhibit reduced estradiol related receptor binding activity, and methods for identifying monomers that exhibit reduced estradiol related receptor binding activity. This abstract is intended as a scanning tool for purposes of searching in the particular art and is not intended to be limiting of the present disclosure.

### BACKGROUND

Development of alternatives to existing polycarbonates and polyphenols that maintain properties (low cost, high transparency and good melt stability) of the corresponding polymers are of great interest in the plastics industry and for the manufacturing industry. To achieve this, suitable monomers for polymerization reactions are necessary to produce polymeric materials with the necessary properties.

Further, monomers or oligomers used in making the polymeric materials may not proceed to completion in some instances, thus leading to the presence of unreacted residual monomers or oligomers in the polymeric material. Additionally, when subjected to certain conditions, the polymeric materials can undergo degradation reactions, such as hydrolytic or thermolytic degradation, resulting in the formation of hydrolysis and/or thermolysis degradants or reaction products. In some aspects, the resulting degradants can correspond chemically to the monomeric starting materials initially used to manufacture polymeric materials. The presence of residual monomers, either as residues of polymerization or through degradation by thermal or hydrolytic means, is an area of growing regulatory concern.

This concern has led to extensive research to find suitable alternative monomers for polymeric materials whose residual monomers or degradation products exhibit desirable characteristics. Desirable characteristics of such degradants include, among other, extremely low, or even no estradiol binding activity.

Accordingly, there remains a need for alternative monomers and polymeric materials which have extremely low, or even no estradiol binding activity. Furthermore, the down-selecting of the candidate monomers, further synthesis and bio-assay testing of candidate monomers is extremely time consuming and expensive. Hence, there also remains a need for developing alternative methods for designing and/or formulating alternative monomers for synthesis and binding activity testing.

### SUMMARY

In various aspects, the present disclosure relates to monomers that exhibit reduced estradiol related receptor binding activity, and methods for identifying monomers that exhibit reduced estradiol related receptor binding activity.

In one aspect, the disclosure relates to a method for determining relative binding energy of a monomer, the method comprising: a) determining a first binding energy (BE) of an a phenolic monomer having alkyl or oxygenated substituents, preferably an alkoxy or acid substituted bisphenol monomer; b) determining a second binding energy (BE) of the corresponding unsubstituted reference monomer; and c) determining the relative binding energy (RBE) based on the first binding energy and second binding energy.

In another aspect, the disclosure relates to a method for determining relative binding energy of monomers having reduced or no estradiol related receptor binding activity, the method comprising: a) providing at least one a phenolic monomer having alkyl or oxygenated substituents, preferably an alkoxy or acid substituted bisphenol monomer; b) determining the binding energy (BE) of the substituted monomer using a computing device; c) determining the binding energy (BE) of the corresponding unsubstituted reference monomer using a computing device; and d) determining the relative binding energy (RBE) of the substituted monomer to the unsubstituted corresponding reference monomer.

In various further aspects, the disclosure relates to methods of making polymeric compositions using the disclosed methods and monomers.

In various further aspects, the disclosure relates to a method for preparing a polymeric composition, the method comprising: a) determining a first binding energy of a phenolic monomer having alkyl or oxygenated substituents, preferably an alkoxy or acid substituted bisphenol monomer, to an estradiol related; b) determining a second binding energy of the corresponding unsubstituted reference monomer to the estradiol related receptor; c) determining the relative binding energy based on the first binding energy and second binding energy; and d) reacting the alkoxy or acid substituted monomer under conditions effective to provide a polymeric composition if the alkoxy or acid substituted monomer exhibits a relative binding energy in the range of from 2.5 kcal/mol to 14 kcal/mol or an intra-molecular hydrogen bond in a range of from 1.7 to 2.08 Å; wherein the polymeric composition does not exhibit a half maximal inhibitory concentration (IC₅₀) less than 0.00025M for ERR α or β in vitro estradiol receptors.

In various further aspects, the disclosure relates to a method for preparing a polymeric composition, the method comprising: a) providing at least one phenolic monomer having alkyl or oxygenated substituents, preferably an alkoxy or acid substituted bisphenol monomer; b) determining the binding energy (BE) of the substituted monomer using a computing device; c) determining the binding energy (BE) of the corresponding unsubstituted reference monomer using a computing device; and d) determining the relative binding energy (RBE) of the substituted monomer to the unsubstituted corresponding reference monomer; and e) reacting the substituted monomer under conditions effective to provide a polymeric composition when the substituted monomer exhibits an RBE in the range of from about 2.5 kcal/mol to about 14 kcal/mol or an intramolecular bond length in a range of from 1.7 to 2.08 Å. In some aspects, the polymeric compositions may be capped at one or more ends with optionally substituted phenols.

In various further aspects, the disclosure relates to articles comprising the disclosed compositions.

While aspects of the present disclosure can be described and claimed in a particular statutory class, such as the system statutory class, this is for convenience only and one of skill in the art will understand that each aspect of the present disclosure can be described and claimed in any statutory class. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

Additional aspects of the disclosure will be set forth in part in the description which follows, and in part will be obvious from the description, or can be learned by practice of the disclosure. The advantages of the disclosure will be realized and attained by means of the elements and combinations particularly pointed out in the appended claims. It is to be understood that both the foregoing general description and the following detailed description are exemplary and explanatory only and are not restrictive of the disclosure, as claimed.

### BRIEF DESCRIPTION OF THE FIGURES

The accompanying figures, which are incorporated in and constitute a part of this specification, illustrate several aspects and together with the description serve to explain the principles of the disclosure.

FIG. 1 shows the crystal structure of the 17β estradiol in the ligand binding domain (LBD) cavity of estradiol related receptor-α (ERR-α).

FIG. 2 shows the structure and inter-molecular bonds (dotted lines) of the 17β estradiol with the amino acids of the ligand binding domain (LBD) cavity and water molecule of estradiol related receptor-α (ERR-α).

FIG. 3A shows representative intra-molecular hydrogen bonding of the monomers according to the present disclosure. The Y¹ and Z substituents are described in the description herein. FIG. 3B shows a more specific model where Z is hydroxycarbonyl (see text)

FIG. 4 shows inter and intra-molecular hydrogen bonding of 5,5′ methylene bis (2-hydroxy benzoic acid) (MdSA) with the amino acid of the ligand binding domain (LBD) cavity of ERR-α.

FIG. 5 shows a block diagram illustrating an exemplary computer operating environment for performing certain aspects of the disclosed methods.

FIG. 6 shows a schematic flowchart showing one aspect of the present disclosure.

### DETAILED DESCRIPTION

The present disclosure can be understood more readily by reference to the following detailed description of the disclosure and the examples included therein.

Before the present compounds, compositions, articles, systems, devices, and/or methods are disclosed and described, it is to be understood that they are not limited to specific synthetic methods unless otherwise specified, or to particular reagents unless otherwise specified, as such can, of course, vary. It is also to be understood that the terminology used herein is for the purpose of describing particular aspects only and is not intended to be limiting. Although any methods and materials similar or equivalent to those described herein can be used in the practice or testing of the present disclosure, example methods and materials are now described.

Moreover, it is to be understood that unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; and the number or type of aspects described in the specification.

All publications mentioned herein are incorporated herein by reference to disclose and describe the methods and/or materials in connection with which the publications are cited.

### A. DEFINITIONS

It is also to be understood that the terminology used herein is for the purpose of describing particular aspects only and is not intended to be limiting. As used in the specification and in the claims, the term “comprising” can include the aspects “consisting of” and “consisting essentially of” Unless defined otherwise, all technical and scientific terms used herein have the same meaning as commonly understood by one of ordinary skill in the art to which this disclosure belongs. In this specification and in the claims which follow, reference will be made to a number of terms which shall be defined herein.

As used in the specification and the appended claims, the singular forms “a,” “an” and “the” include plural referents unless the context clearly dictates otherwise. Thus, for example, reference to “a polymer” includes mixtures of two or more polymers.

As used herein, the term “combination” is inclusive of blends, mixtures, alloys, reaction products, and the like.

Ranges can be expressed herein as from one particular value, and/or to another particular value. When such a range is expressed, another aspect includes from the one particular value and/or to the other particular value. Similarly, when values are expressed as approximations, by use of the antecedent ‘about,’ it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, the terms “about” and “at or about” mean that the amount or value in question can be the value designated some other value approximately or about the same. It is generally understood, as used herein, that it is the nominal value indicated ±10% variation unless otherwise indicated or inferred. The term is intended to convey that similar values promote equivalent results or effects recited in the claims. That is, it is understood that amounts, sizes, formulations, parameters, and other quantities and characteristics are not and need not be exact, but can be approximate and/or larger or smaller, as desired, reflecting tolerances, conversion factors, rounding off, measurement error and the like, and other factors known to those of skill in the art. In general, an amount, size, formulation, parameter or other quantity or characteristic is “about” or “approximate” whether or not expressly stated to be such. It is understood that where “about” is used before a quantitative value, the parameter also includes the specific quantitative value itself, unless specifically stated otherwise.

As used herein, the terms “optional” or “optionally” means that the subsequently described event or circumstance can or cannot occur, and that the description includes instances where said event or circumstance occurs and instances where it does not. For example, the phrase “optionally substituted alkyl” means that the alkyl group can or cannot be substituted and that the description includes both substituted and unsubstituted alkyl groups.

As used herein, the term “effective amount” refers to an amount that is sufficient to achieve the desired modification of a physical property of the composition or material. For example, an “effective amount” of a filler refers to an amount that is sufficient to achieve the desired improvement in the property modulated by the formulation component, e.g. achieving the desired level of modulus. The specific level in terms of wt % in a composition required as an effective amount will depend upon a variety of factors including the amount and type of polycarbonate, amount and type of polycarbonate, amount and type of thermally conductive filler, and end use of the article made using the composition.

Disclosed are the components to be used to prepare the compositions of the disclosure as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds cannot be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C—F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the compositions of the disclosure. Thus, if there are a variety of additional steps that can be performed it is understood that each of these additional steps can be performed with any specific aspect or combination of aspects of the methods of the disclosure.

References in the specification and concluding claims to parts by weight of a particular element or component in a composition or article, denotes the weight relationship between the element or component and any other elements or components in the composition or article for which a part by weight is expressed. Thus, in a compound containing 2 parts by weight of component X and 5 parts by weight component Y, X and Y are present at a weight ratio of 2:5, and are present in such ratio regardless of whether additional components are contained in the compound.

As used herein the terms “weight percent,” “wt %,” and “wt. %,” which can be used interchangeably, indicate the percent by weight of a given component based on the total weight of the composition, unless otherwise specified. That is, unless otherwise specified, all wt % values are based on the total weight of the composition. It should be understood that the sum of wt % values for all components in a disclosed composition or formulation are equal to 100.

Compounds are described using standard nomenclature. For example, any position not substituted by any indicated group is understood to have its valence filled by a bond as indicated, or a hydrogen atom. A dash (“-”) that is not between two letters or symbols is used to indicate a point of attachment for a substituent. For example, —CHO is attached through carbon of the carbonyl group. Unless defined otherwise, technical and scientific terms used herein have the same meaning as is commonly understood by one of skill in the art to which this disclosure belongs.

The term “alkyl group” as used herein is a branched or unbranched saturated hydrocarbon group of 1 to 24 carbon atoms, such as methyl, ethyl, n propyl, isopropyl, n butyl, isobutyl, t butyl, pentyl, hexyl, heptyl, octyl, decyl, tetradecyl, hexadecyl, eicosyl, tetracosyl and the like. A “lower alkyl” group is an alkyl group containing from one to six carbon atoms.

The term “aryl group” as used herein is any carbon-based aromatic group including, but not limited to, benzene, naphthalene, etc. The term “aromatic” also includes “heteroaryl group,” which is defined as an aromatic group that has at least one heteroatom incorporated within the ring of the aromatic group. Examples of heteroatoms include, but are not limited to, nitrogen, oxygen, sulfur, and phosphorus. The aryl group can be substituted or unsubstituted. The aryl group can be substituted with one or more groups including, but not limited to, alkyl, alkynyl, alkenyl, aryl, halide, nitro, amino, ester, ketone, aldehyde, hydroxy, carboxylic acid, or alkoxy.

The term “aralkyl” as used herein is an aryl group having an alkyl, alkynyl, or alkenyl group as defined above attached to the aromatic group. An example of an aralkyl group is a benzyl group.

The term “carbonate group” as used herein is represented by the formula OC(O)OR, where R can be hydrogen, an alkyl, alkenyl, alkynyl, aryl, aralkyl, cycloalkyl, halogenated alkyl, or heterocycloalkyl group described above.

The term “organic residue” defines a carbon containing residue, i.e., a residue comprising at least one carbon atom, and includes but is not limited to the carbon-containing groups, residues, or radicals defined hereinabove. Organic residues can contain various heteroatoms, or be bonded to another molecule through a heteroatom, including oxygen, nitrogen, sulfur, phosphorus, or the like. Examples of organic residues include but are not limited alkyl or substituted alkyls, alkoxy or substituted alkoxy, mono or di-substituted amino, amide groups, etc. Organic residues can preferably comprise 1 to 18 carbon atoms, 1 to 15, carbon atoms, 1 to 12 carbon atoms, 1 to 8 carbon atoms, 1 to 6 carbon atoms, or 1 to 4 carbon atoms. In a further aspect, an organic residue can comprise 2 to 18 carbon atoms, 2 to 15, carbon atoms, 2 to 12 carbon atoms, 2 to 8 carbon atoms, 2 to 4 carbon atoms, or 2 to 4 carbon atoms.

A very close synonym of the term “residue” is the term “radical,” which as used in the specification and concluding claims, refers to a fragment, group, or substructure of a molecule described herein, regardless of how the molecule is prepared. For example, a 2,4-dihydroxyphenyl radical in a particular compound has the structure:

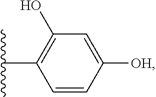 [SMILES: *c1ccc(O)cc1O]

regardless of whether 2,4-dihydroxyphenyl is used to prepare the compound. In some aspects the radical (for example an alkyl) can be further modified (i.e., substituted alkyl) by having bonded thereto one or more “substituent radicals.” The number of atoms in a given radical is not critical to the present disclosure unless it is indicated to the contrary elsewhere herein.

“Organic radicals,” as the term is defined and used herein, contain one or more carbon atoms. An organic radical can have, for example, 1-26 carbon atoms, 1-18 carbon atoms, 1-12 carbon atoms, 1-8 carbon atoms, 1-6 carbon atoms, or 1-4 carbon atoms. In a further aspect, an organic radical can have 2-26 carbon atoms, 2-18 carbon atoms, 2-12 carbon atoms, 2-8 carbon atoms, 2-6 carbon atoms, or 2-4 carbon atoms. Organic radicals often have hydrogen bound to at least some of the carbon atoms of the organic radical. One example, of an organic radical that comprises no inorganic atoms is a 5,6,7,8-tetrahydro-2-naphthyl radical. In some aspects, an organic radical can contain 1-10 inorganic heteroatoms bound thereto or therein, including halogens, oxygen, sulfur, nitrogen, phosphorus, and the like. Examples of organic radicals include but are not limited to an alkyl, substituted alkyl, cycloalkyl, substituted cycloalkyl, mono-substituted amino, di-substituted amino, acyloxy, cyano, carboxy, carboalkoxy, alkylcarboxamide, substituted alkylcarboxamide, dialkylcarboxamide, substituted dialkylcarboxamide, alkylsulfonyl, alkylsulfinyl, thioalkyl, thiohaloalkyl, alkoxy, substituted alkoxy, haloalkyl, haloalkoxy, aryl, substituted aryl, heteroaryl, heterocyclic, or substituted heterocyclic radicals, wherein the terms are defined elsewhere herein. A few non-limiting examples of organic radicals that include heteroatoms include alkoxy radicals, trifluoromethoxy radicals, acetoxy radicals, dimethylamino radicals and the like.

As used herein, the terms “number average molecular weight” or “Mₙ” can be used interchangeably, and refer to the statistical average molecular weight of all the polymer chains in the sample and is defined by the formula:

$${M}_{n} = \frac{Σ {N}_{i} {M}_{i}}{Σ {N}_{i}} ,$$

where Mᵢ is the molecular weight of a chain and Nᵢ is the number of chains of that molecular weight. Mₙ can be determined for polymers, e.g., polycarbonate polymers, by methods well known to a person having ordinary skill in the art using molecular weight standards, e.g. polycarbonate standards or polystyrene standards, preferably certified or traceable molecular weight standards.

As used herein, the terms “weight average molecular weight” or “Mw” can be used interchangeably, and are defined by the formula:

$${M}_{w} = \frac{Σ {N}_{i} {M}_{i}^{2}}{Σ {N}_{i} {M}_{i}} ,$$

where Mᵢ is the molecular weight of a chain and Nᵢ is the number of chains of that molecular weight. Compared to Mₙ, M_{w} takes into account the molecular weight of a given chain in determining contributions to the molecular weight average. Thus, the greater the molecular weight of a given chain, the more the chain contributes to the M_{w}. M_{w} can be determined for polymers, e.g. polycarbonate polymers, by methods well known to a person having ordinary skill in the art using molecular weight standards, e.g. polycarbonate standards or polystyrene standards, preferably certified or traceable molecular weight standards.

As used herein, the terms “polydispersity index” or “PDI” can be used interchangeably, and are defined by the formula:

$$P D I = \frac{{M}_{w}}{{M}_{n}} .$$

The PDI has a value equal to or greater than 1, but as the polymer chains approach uniform chain length, the PDI approaches unity.

As used herein, “polycarbonate” refers to an oligomer or polymer comprising residues of one or more dihydroxy compounds, e.g., dihydroxy aromatic compounds, joined by carbonate linkages; it also encompasses homopolycarbonates, copolycarbonates, and (co)polyester carbonates.

The terms “residues” and “structural units”, used in reference to the constituents of the polymers, are synonymous throughout the specification.

As used herein the terms “weight percent,” “wt %,” and “wt. %,” which can be used interchangeably, indicate the percent by weight of a given component based on the total weight of the composition, unless otherwise specified. That is, unless otherwise specified, all wt % values are based on the total weight of the composition. It should be understood that the sum of wt % values for all components in a disclosed composition or formulation are equal to 100.

As used herein, the term half maximal inhibitory concentration (IC₅₀) is a quantitative measure that indicates how much of a particular substance, i.e., an inhibitor, is needed to inhibit a given biological process or component of a process, by one half. In other words, it is the half maximal (50%) inhibitory concentration (IC) of a substance (50% IC, or IC₅₀). It is commonly known to one of ordinary skill in the art and used as a measure of antagonist drug potency in pharmacological research. The (IC₅₀) of a particular substance can be determined using conventional competition binding assays. In this type of assay, a single concentration of radioligand (such as an agonist) is used in every assay tube. The ligand is used at a low concentration, usually at or below its K_{d} value. The level of specific binding of the radioligand is then determined in the presence of a range of concentrations of other competing non-radioactive compounds (usually antagonists), in order to measure the potency with which they compete for the binding of the radioligand. Competition curves can also be computer-fitted to a logistic function as described under direct fit. The IC₅₀ is the concentration of competing ligand which displaces 50% of the specific binding of the radioligand.

Each of the materials disclosed herein are either commercially available and/or the methods for the production thereof are known to those of skill in the art.

It is understood that the compositions disclosed herein have certain functions. Disclosed herein are certain structural requirements for performing the disclosed functions, and it is understood that there are a variety of structures that can perform the same function that are related to the disclosed structures, and that these structures will typically achieve the same result.

### B. METHODS OF DETERMINING INTER AND INTRA-MOLECULAR HYDROGEN BOND LENGTH

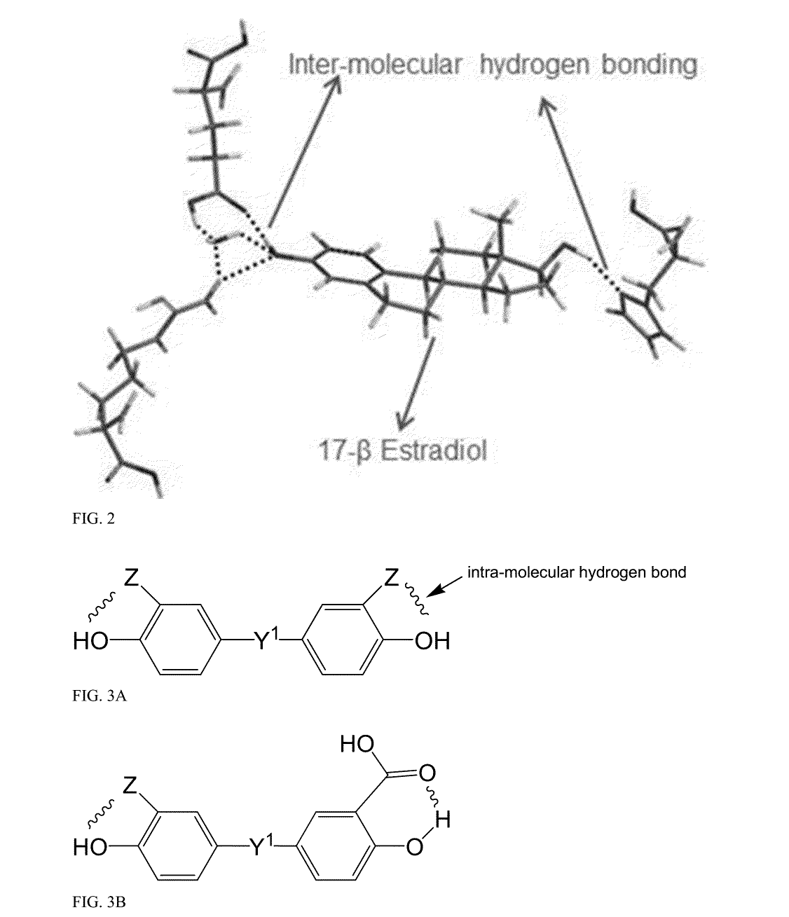

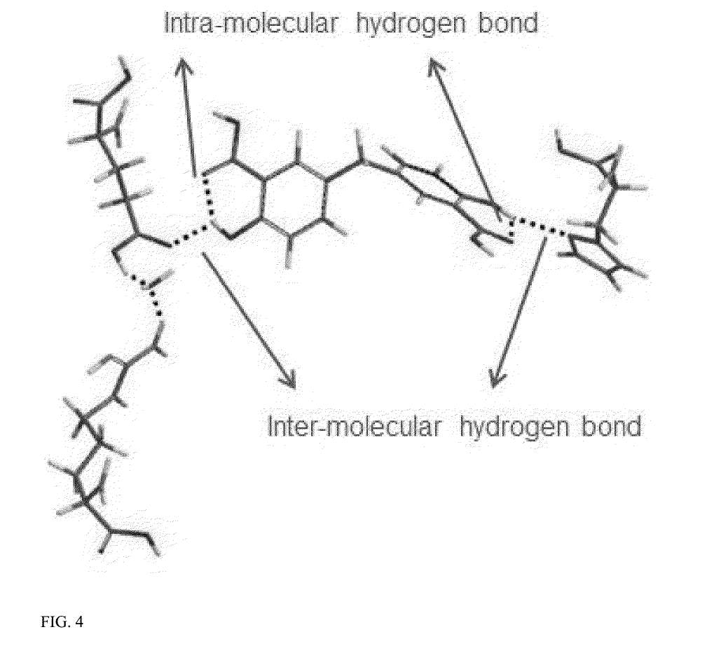

In various aspects, hydrogen bonding plays an important role in biological systems where an electronegative atom, such as, O, N, or S, is attracted by electropositive atoms such as H. For example. hormones present in the biological systems, such as estradiol, are bonded to amino acids of estradiol receptors via hydrogen bonds. In one aspect, the main interaction within the ligand binding domain (LBD) cavity of ERR is the inter-molecular hydrogen bonding with the amino acids of the LBD cavity and the ligand. For example, as shown in FIG. 2, the phenolic hydroxyl group of 17β estradiol can make inter-molecular hydrogen bonds with Glutamine (Glu), water molecule and Argenine (Arg), and stabilizes the molecule in the LBD cavity. On the other side, the alcoholic OH group of the 17β estradiol hydrogen bonds with Histidine (His). Without wishing to be bound by a particular theory, absence of the hydroxyl groups on a ligand can reduce or eliminate inter-molecular hydrogen bonding with the amino acids of the LBD, resulting in the ligand having reduced or no binding activity with the LBD cavity of ERR.

In one aspect, the present method relates to intra-molecular hydrogen bonding of ligands, such as, for example, substituted monomers. In a further aspect, the present methods utilize intra-molecular hydrogen bonding, for example, in the form of molecular descriptors or measurements, in at least one step of the methods.

In one aspect, the intra-molecular hydrogen bond is the hydrogen bond between the hydrogen of a hydroxyl group and an electronegative group. In a further aspect, the intra-molecular hydrogen bond is the hydrogen bond between the hydrogen of a hydroxyl group and an electronegative group, such as, O, N, or S, or an oxygen-containing group, substituted in the ortho position of the same molecule denoted by Z, as shown in FIG. 3A. In certain embodiments, the electronegative group comprises a O, N, or S, but not a halogen or hydroxy. In a still further aspect, the Z group can be any desired group, including, but not limited to carboxylic, keto, ester, ether, aldehyde, or alkoxy. In a yet further aspect, the Y¹ substituent can comprise any desired linear, branched or cyclic group.

In a further aspect, Y¹ can be a substituted or unsubstituted C₃-C₁₂ cycloalkylidene; a C₃-C₁₂ alkylidene of the formula —C(R^{c})(R^{d})—wherein R^{c} and R^{d} are each independently hydrogen, C₁-C₁₂ alkyl, C1-C₁₂ cycloalkyl, C₇-C₁₂ arylalkyl. In yet further aspects, Y¹ is OR wherein R is selected from methyl, ethyl, propyl, octyl, isooctyl, benzyl, ethyl phenyl, butyl phenyl, propyl diphenyl, and cyclohexyl phenyl. In even further aspects, R is methyl. In even further aspects, Z is selected from methylene, cyclohexyl-methylene, 2-[2.2.1]-bicycloheptylidene, ethylidene, isopropylidene, neopentylidene, cyclohexylidene, cyclopentadecylidene, cyclododecylidene, and adamantylidene. In other aspects, Z is selected from ethylidene, isopropylidene, isobutylidene neopentylidene, cyclohexylidene, alkyl substituted cyclohexylidene, aryl, cyclopentadecylidene, cyclododecylidene, sulfo, oxo, and bicycloheptylidene; and wherein Y¹ is OR and each R is selected from methyl, ethylidene, propylidene, butylidene, benzyl, phenyl, C1 to C4 alkyl phenyl, and cyclohexylidene.

In various aspects, the present methods utilize structural data describing the structure of a protein, or a receptor, or a ligand, or combinations thereof. In a further aspect, structural data can be any desired structural data. In one aspect, structural data can comprise a set of three-dimensional coordinates defining atomic positions for each atom or group of atoms in the protein or ligand, for example, a data file in the protein data bank (PDB) format for protein structural information and/or the crystallographic information file (CIF) format used by the Cambridge Structural Database for organic ligands.

In other aspects, ligand structural data can comprise monomer structural data comprising two-dimensional drawings showing molecular connectivity (e.g., as a structure data (*.SD), or smiles or *.cdx file as developed, for example, by ChemDraw software), which can be converted to three-dimensional format (e.g. *.mol, *.xyz, *.spartan) using available computing software known to those of skill in the art. In a further aspect, the structural data can be derived from experimentally-determined data. In other aspects, the structural data can be derived from theoretical or computationally determined data.

In one aspect, the method can comprise designing a three-dimensional molecule, such as, for example, a monomer. In a further aspect, the molecule in three dimensional configuration can be optimized in a gas phase with vacuum using quantum mechanical methods. In a still further aspect, the quantum mechanical method can comprise any desired quantum mechanical method, including, and without limitation, density functional theory (DFT), Hartree-Fock (HF), Møller-Plesset (MP) perturbation theory, Configuration Interaction (CI), Coupled-Cluster (CC), and the like.

In other aspects, a combination of quantum mechanical and molecular mechanics approaches (e.g. QM/MM) can be used. In a further aspect, basis sets used can include, and without limitation, 3-21G, 6-31G, 6-31 G*, 6-31G**, 6-31G*+, 6-31G**++, including various combinations of polarized (*) and diffused (+) functions. In a still further aspect, other basis sets with effective core, such as, for example LACVP, can be used in combination with the complete basis sets. In a further aspect, several exchange correlation functions can be used in DFT approaches, such as, and without limitation, local density approximation (LDA) or generalized gradient approximation (GGA), for example, B3LYP, PBE, BLYP, PW91, TPSS, TPSSh, LHFLYP, COHSEX, and the like.

In one aspect, the computational simulations can be performed by the DFT approach using 6-31G* basis set and B3-LYP exchange-correlation functional. In a further aspect, the computational simulations can be performed using commercially available quantum mechanical simulation software, such as, for example, SPARTAN from WAVEFUNCTION, Inc. of Irvine, Calif.

### C. METHODS OF DETERMINING BINDING ENERGY

In various aspects, the present disclosure relates to methods and systems for predicting binding energy. In further aspects, the present disclosure relates to methods for predicting the expected binding energy between molecular entities, such as a monomer (e.g., a ligand) and a protein (e.g., LBD of ERR).

In one aspect, the binding energy is a relative binding energy (RBE). In a further aspect, relative binding energy (RBE) is the difference in the calculated binding energies (BE) between a substituted monomer and its corresponding unsubsubstituted monomer as described herein. In a still further aspect, the corresponding monomer is a substantially identical reference monomer without the substituted group. For example, for a methoxy substituted monomer, the corresponding monomer would be a substantially identical monomer without the methoxy group.

In a further aspect, the method comprises the steps of: a) determining a first binding energy (BE) of a substituted monomer; b) determining a second binding energy (BE) of the corresponding unsubstituted reference monomer; and c) determining the relative binding energy (RBE) based on the first binding energy and second binding energy.

In a further aspect, the method comprises the steps of: a) providing at least one substituted monomer; b) determining the binding energy (BE) of the substituted monomer using a computing device; c) determining the binding energy (BE) of the corresponding unsubstituted reference monomer using a computing device; and d) determining the relative binding energy (RBE) of the substituted monomer to the unsubstituted corresponding reference monomer. In certain aspects, the substituted monomers comprise alkyl or oxgenated substituents, as described elsewhere herein. In other aspects, the substituted monomers comprise an alkoxy or carboxylic acid substituent.

In one aspect, the relative binding energy is the binding energy difference to an estradiol related receptor (ERR) between a monomer with at least one intra-molecular hydrogen bond and the respective corresponding reference monomer without the intra-molecular hydrogen bond. In a further aspect, the estradiol related receptor comprises ERR-α, ERR-β, or ERR-γ, or a combination thereof. In some aspects, the estradiol related receptor is ERR-α. In other aspects, the estradiol related receptor is ERR-β. In a further aspect, the binding energy (BE) of a monomer is calculated to the ligand binding domain (LBD) of an estradiol related receptor (ERR), including ERR-α.

In further aspects, the present disclosure relates to determining the relative binding energy (RBE) of monomers having an intra-molecular hydrogen bond in the LBD cavity of estradiol related receptors (ERR) compared to the binding energy of monomers without the intra-molecular hydrogen bond in the LBD cavity of estradiol related receptors (ERR). In some aspects, the estradiol related receptors can comprise ERR-α receptors. In other aspects, the estradiol related receptors can comprise ERR-β receptors. In further aspects, the estradiol related receptors can comprise ERR-γ receptors.

In further aspects, the present disclosure provides a method for determining the variation in the relative binding energy of monomers based on the binding energies in the LBD cavity of estradiol related receptors using molecular modeling data based on quantum mechanical methodology as described herein. In other aspects, monomer or ligand structural data can comprise two-dimensional drawings showing molecular connectivity (e.g., as a structure data (*.SD) file or smiles), which can be converted to three-dimensional format using available computing software known to those of skill in the art. In a further aspect, the structural data can be derived from experimentally-determined data. In other aspects, the structural data can be derived from theoretical or computationally determined data.

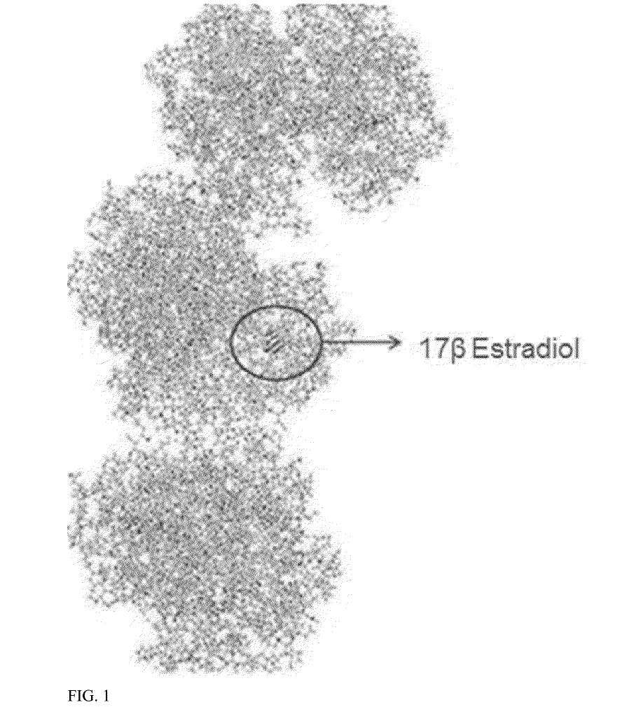

In one aspect, the methods utilize structural data for a ligand binding domain (LBD) cavity of a receptor. In a further aspect, the receptor is ERR-α, and the binding domain is the LBD cavity of ERR-α, as shown in FIG. 1 and FIG. 2.

In various aspects, binding modes can be described using the amino acids that make direct electrostatic or van der Waal's contact with, or form intermolecular hydrogen bonds with the ligand. In one aspect, the distances between critical amino acids that contribute to the binding of the ligand, and the bound ligand can be measured to generate a distance map. In a further aspect, the distance map can be used according to known techniques to provide a geometric model representing features that can be required for or effect binding with the protein. In still further aspect, the method can comprise using the geometric model as an input to perform computer simulations to predict the binding energy of a monomer, for example, the relative binding energy of the monomer.

In further aspects, a number of binding conformations can exist for any given monomer or ligand in the ligand binding domain. In one aspect, the method comprises optimizing the binding conformations of the ligand or monomer. In one aspect, the binding confirmations can be optimized using any desired technique or method. In a further aspect, any suitable technique for determining the energetically favored conformation between two interacting molecules can be used. In one aspect, the binding conformations of the protein or amino acids which are binding the ligands are not optimized. In a further aspect, the binding conformations of the protein or amino acids are fixed. In a further aspect, the energy of the protein or receptor corresponds to the unoptimized energy of the LBD cavity.

In a further aspect, the method comprises determining a binding energy for each ligand or monomer in the corresponding optimized binding conformations. In a still further aspect, the monomer is optimized to the lowest configuration attained in the fixed LBD configuration. In a yet further aspect, the monomer is optimized for the lowest calculated binding energy in the optimized binding conformation. In some aspects, the calculated binding energies comprise the predicted binding energies for each of the monomers or ligands. In further aspects, the lowest energy conformation for the monomer or ligand is selected and binding energies are calculated for the monomer in that conformation.

In one aspect, the method can comprise any number of molecular measurements or descriptors for use in predicting binding energy or binding affinity. In one aspect, larger numbers of measurements or descriptors can result in complex and difficult prediction model, requiring substantial computational power and time, for example, when calculating binding energies of the monomers in the LBD cavity that includes the complete protein.

In one aspect, the present methods utilize the constrained geometry optimization approach for calculating the binding energy. In a further aspect, the active amino acids (FIG. 2) which are binding to the ligands are not optimized. In a still further aspect, amino acid coordinates are fixed and are derived from experimental data where the ligand binding domain (LBD) cavity of the amino acid is truncated by substituting hydrogen atoms to neutralize the valencies. In a yet further aspect, the monomer coordinates are completely optimized to the lowest configuration attained in a fixed LBD cavity configuration.

In one aspect, the binding energy (BE) value is determined using the formula: BE=Energy (complex)−[Energy(cavity)+Energy(monomer)], where the energy corresponds to electronic energy of the system—e.g., monomer, cavity, and complex. In a further aspect, the energies of the cavity and monomer are calculated in the gas phase in vacuum. In a still further aspect, the complex described in the above equation corresponds to the energy of the optimized structure of the monomer in the constrained structure of the cavity. In some aspects, the calculations are performed using density functional theory (DFT) approach with 6-31G* basis set and B3-LYP functional as described above. In a further aspect, relative binding energy (RBE) is the difference in the calculated binding energies (BE) between a substituted monomer and its corresponding unsubsubstituted reference monomer as described herein, and can be determined using the formula: RBE=BE (substituted monomer)−BE (unsubstituted monomer).

As described above, the present methods, in various aspects, use calculated ligand-receptor binding energies. In further aspects, the calculated ligand-receptor binding energies can depend on the mode and nature of the ligand-receptor interaction. In a further aspect, the binding energies selected are related to the strength of binding of the monomers to the ligand binding domain (LBD) cavity.

In some aspects, the relationship between the calculated BE of selected monomer-receptor complex systems and the experimentally determined IC₅₀ values of the monomer cannot be correlated or are non-linear. In other aspects, the binding energy values can be used for predicting the relative binding behavior of the monomers to the LBD cavity of estradiol related receptors. In further aspects, the binding energy values can be used to predict the relative increase or decrease of the binding activity of the monomers to the LBD cavity of ERR.

In one aspect, the method can comprise correlating between binding behavior and at least one molecular or atomic feature of interacting molecules. In a further aspect, the feature can be an inter-molecular or an intra-molecular feature, for example, intra-molecular hydrogen bonding of the monomer.

In one aspect, determining the relative binding energy of the monomer comprises estimating the difference in binding energies of the monomers. In a further aspect, the relative binding energy is determined by comparing the calculated binding energy of the monomer with the binding energy of a known reference compound calculated in the same manner for example the relative binding energy is the difference in the binding energy of the monomer with the intra-molecular hydrogen bond and the monomer without the intra-molecular hydrogen bond. In a still further aspect, the relative binding energy is used to determine binding behavior of previously tested or untested monomers to ERR. In a yet further aspect, the relative binding energy permits determination of the ERR binding activity from untested monomers with respect to a tested reference monomer.

In one aspect, the monomer comprises at least one intra-molecular hydrogen bond having a length in the range of from about 1.6 to about 3.0 Å, for example, in the range of from about 1.7 to about 2.1 Å

In a yet further aspect, the relative binding energy values can thus be used to determine if monomers possessing intra-molecular hydrogen bonding have reduced binding activity with respect to corresponding reference monomers without intra-molecular hydrogen bonding can be suitable for use in the manufacture of polymeric compositions exhibiting reduced or no estradiol binding activity. For example, the method can further comprise comparing the determined relative binding energy of at least one monomer to a predefined relative binding energy to determine whether the determined relative binding energy satisfies the predefined standard relative binding energy, wherein when the determined relative binding energy satisfies the predefined standard relative binding energy, the untested monomer can be used to produce polymeric compositions that exhibit reduced or no estradiol activity, for example, that does not exhibit a half maximal inhibitory concentration (IC₅₀) less than 0.00025M for alpha or beta in vitro estradiol receptors.

Thus, according to other aspects, the methods can further comprise the step of reacting the monomer under conditions effective to provide a polymeric composition when the relative binding energy of the substituted monomer possessing intra-molecular hydrogen bond is greater than 0 kcal/mol. In a further aspect, the relative binding energy of the monomer is at least about 0.5, 1.0, 1.5, 2.0, or 2.5 kcal/mol. In a still further aspect, the relative binding energy of the monomer is in a range from greater than 0 kcal/mol to about 25 kcal/mol, for example, in the range of from about 2.7 kcal/mol to about 14 kcal/mol.

The disclosed methods can be applied to a large number of monomer candidates. In one aspect, the present methods can be used to select those monomers with a desirable predicted binding energy for further evaluation, thus reducing the time and effort required to identify promising monomers. In another aspect, the present methods for determining relative binding energy can be used to computationally evaluate the expected estradiol relative binding activity of a candidate monomer with respect to a reference monomer.

In further aspects, at least one step of the disclosed methods is performed by a computing device. In one aspect, the computing device can comprise a computing system. In a further system, the computing system generally comprises computing hardware and computing software for performing various computing tasks or instructions, for example, molecular modeling and analysis of data. In a still further aspect, the computing software can comprise any desired molecular modeling program. Those of skill in the art will recognize that the computing system can be configured in a number of ways using known computing hardware and known computing software, such as, for example, Spartan software from Wavefunction Inc. as described herein.

In a further aspect, the binding energies for different monomers or ligands can be compared and ordered to identify those monomers having the desirable level of binding affinity for the receptor. In some aspects, the binding energy values can be compared to experimental-determined binding affinity data, for example, IC₅₀, for the monomers or a subset thereof. In other aspects, the binding energy values can be compared to calculated binding activity data of related monomers for the monomers or a subset thereof.

In one aspect, the present disclosure relates to computing program products including machine-readable media on which are provided computing program instructions or code for carrying out at least one step of the disclosed methods. In a further aspects, methods of the present disclosure may be represented, in whole or in part, as computing program instructions or code that can be provided as an executable file on such machine-readable media. In a still further aspect, the disclosure relates and can comprise combinations and arrangements of data generated and/or used as described herein.

In some aspects, the monomers comprise bisphenol based and substituted bisphenol based monomers. In one aspect, the monomer can comprise an aromatic organic radical and, more preferably, a radical of the formula (2):

-A¹-Y¹-A²- (2),

wherein each of A¹ and A² is a monocyclic divalent aryl radical and Y¹ is a bridging radical having one or two atoms that separate A¹ from A². In various aspects, one atom separates A¹ from A². For example, radicals of this type include, but are not limited to, radicals such as —O—, —S—, —S(O)—, —S(O₂)—, —C(O)—, methylene, cyclohexyl-methylene, 2-[2.2.1]-bicycloheptylidene, ethylidene, isopropylidene, neopentylidene, cyclohexylidene, cyclopentadecylidene, cyclododecylidene, and adamantylidene. The bridging radical Y¹ is preferably, but not necessarily, a hydrocarbon group or a saturated hydrocarbon group such as methylene, cyclohexylidene, or isopropylidene. In some aspects, the monomers comprise dihydroxy compounds having the formula HO—R¹—OH, which includes dihydroxy compounds of formula (3):

HO-A¹-Y¹-A²-OH (3),

wherein Y¹, A¹ and A² are as described above. Each of the structures or spatial arrangements shown in Table 1 are aspects of the present disclosure.

Other aspects include monomer compounds having a general formula (4A) or (4B):

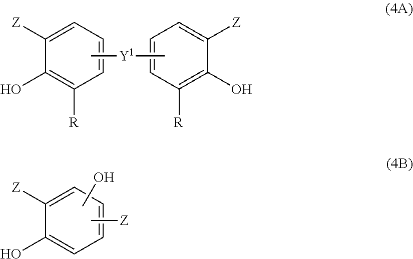

where Y¹ is as defined above, where R is independently at each occurrence an H, C₁₋₁₈ alkyl, C₆₋₁₈-aryl, or C₇₋₁₈-alkylaryl group and Z is independently at each occurrence H or an oxygenated substituent of a structure —OR″, —COOR (carboxylic acid or ester), —O—C(O)R (carboxylate), —O—C(O)—OR (carbonate), or —C(O)—R (aldehyde or ketone), where —OR″ is a C₁₋₁₈ alkyl, C₆₋₁₈-aryl, or C₇₋₁₈-alkylaryl alkoxide. In an aspect, the bishydroxy groups are not ortho to one another. Exemplary structures include, but are not limited to:

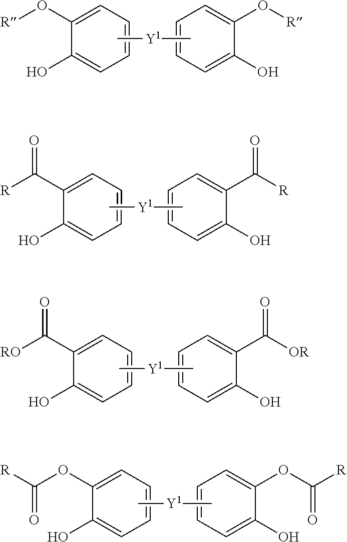

Still other aspects include monomer compounds having a general formula (5):

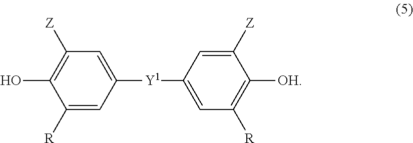

where Y¹, R, and Z are each independently as defined above.

In some aspects, R is H or C₁₋₆ alkyl. In other aspects, R is H or C₁₋₃ alkyl. In still other aspects, R is H or —CH₃.

In some aspects, R″ is C₁₋₁₂ alkyl, C₁₋₆ alkyl, C₁₋₃ alkyl, or —CH₃.

In some aspects, Y¹ is C₃₋₁₂ alkyl (including linear, branched, or cycloalkyl) or aryl. In other aspects, Y¹ is —O—, —S—, —S(O)₂—, or keto (C═O).

As used herein, the term “unsubstituted monomer” or “unsubstituted reference monomer” refers to the case where Z and R are H at every occurrence.

Where phenolic end caps are employed, an end cap monomer of formula (6):

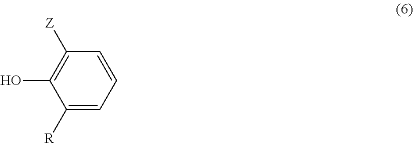

may be employed, where R and Z are as defined herein.

Also included are bisphenol compounds of general formula (7):

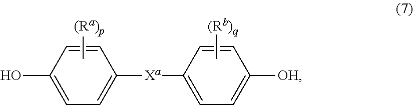

wherein R^{a} and R^{b} each represent a halogen atom or a monovalent hydrocarbon group and can be the same or different; p and q are each independently integers from 0 to 4; and X^{a} represents one of the groups of formula (8):

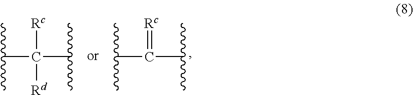

wherein R^{c} and R^{d} each independently represent a hydrogen atom or a monovalent linear or cyclic hydrocarbon group and Re is a divalent hydrocarbon group.

In various aspects, a heteroatom-containing cyclic alkylidene group comprises at least one heteroatom with a valency of 2 or greater, and at least two carbon atoms. Heteroatoms for use in the heteroatom-containing cyclic alkylidene group include —O—, —S—, and —N(Z″)—, where Z″ is a substituent group selected from hydrogen, hydroxy, C₁₋₁₂ alkyl, C₁₋₁₂ alkoxy, or C₁₋₁₂ acyl. Where present, the cyclic alkylidene group or heteroatom-containing cyclic alkylidene group can have 3 to 20 atoms, and can be a single saturated or unsaturated ring, or fused polycyclic ring system wherein the fused rings are saturated, unsaturated, or aromatic.

In various further aspects, the monomer can comprise bisphenols containing substituted or unsubstituted cyclohexane units, for example bisphenols of formula (9):

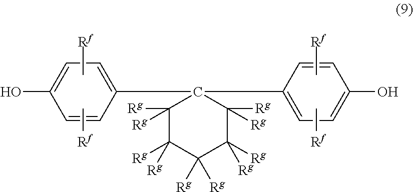

wherein each R^{f} is independently hydrogen, C₁₋₁₂ alkyl, or halogen; and each R^{g} is independently hydrogen or C₁₋₁₂ alkyl. The substituents can be aliphatic or aromatic, straight chain, cyclic, bicyclic, branched, saturated, or unsaturated.

In further aspects, the monomer can comprise a dihydroxy compound having the formula HO—R¹—OH, including aromatic dihydroxy compounds of formula (10):

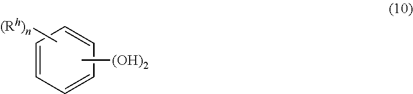

wherein each R^{h} is independently a halogen atom, a C₁₋₁₀ hydrocarbyl such as a C₁₋₁₀ alkyl group, a halogen substituted C₁₋₁₀ hydrocarbyl such as a halogen-substituted C₁₋₁₀ alkyl group, and n is 0 to 4. The halogen is usually bromine.

In a further aspect, the present disclosure provides monomers that do not exhibit significant estradiol-like binding activity. In a further aspect, the lack of significant estradiol like binding activity of the monomers can be characterized by a determination of their half maximal inhibitory concentration (IC₅₀) for ERR-α, ERR-β, or ERR-γ in vitro estradiol receptors. For example, monomers of the present disclosure do not exhibit a half maximal inhibitory concentration (IC₅₀) less than 0.00025M for alpha or beta or gamma in vitro estradiol receptors. In further aspects, monomers of the present disclosure do not exhibit a half maximal inhibitory concentration (IC₅₀) less than 0.0003M, 0.00035M, 0.0004M, 0.00045M, 0.0005M, 0.00075M, or even 0.001M, for alpha or beta or gamma in vitro estradiol receptors. In still further aspects, monomers of the present disclosure do not exhibit any identifiable half maximal inhibitory concentration (IC₅₀) greater than or equal to about 0.00025M, 0.0003M, 0.00035M, 0.0004M, 0.00045M, 0.0005M, 0.00075M, or even 0.001M, for alpha or beta or gamma in vitro estradiol receptors.

It is noted here that, O—O distances between ortho substituents, by themselves, are not necessarily good indicators of estradiol-like binding activity. For example, where other researchers have observed that most strong-binding estrogen receptor ligands may contain two —OH groups with an O—O distance ranging from 9 to 13 Å, the corollary is not necessarily true—i.e., bisphenols having two hydroxy groups having this O—O spacing can still have reduced binding or no binding activity.

### D. METHODS OF PREPARING POLYMERIC COMPOSITIONS

As described above, the present disclosure also relates to methods of making a polymeric composition. Such polymer compositions may include polycarbonates, polyacrylates, epoxides, polysulfones, polyetherimides, and polyurethanes, or any copolymer or blend thereof.

In one aspect, the present disclosure provides a method for preparing a polymeric composition, the method comprising: a) determining a first binding energy of a monomer substituted with an oxygenated substituent to an estradiol related receptor, wherein the monomer comprises a bisphenol monomer, optionally substituted with at least one alkoxy, hydroxycarbonyl, alkoxycarbonyl, carboxylate, carbonate, aldehyde, or ketone; b) determining a second binding energy of the corresponding unsubstituted reference monomer to the estradiol related receptor; c) determining the relative binding energy based on the first binding energy and second binding energy; and d) reacting the monomer substituted with an oxygenated substituent under conditions effective to provide a polymeric composition if the substituted monomer exhibits a relative binding energy in the range of from 2.7 kcal/mol to 14 kcal/mol or an intra-molecular hydrogen bond in a range of from about 1.7 to about 2.08 Å obtained from the method described herein (and as described in Table 2); wherein the polymeric composition does not exhibit a half maximal inhibitory concentration (IC₅₀) less than 0.00025M for ERR α or ERR β in vitro estradiol receptors.

In another aspect, the present disclosure provides a method for preparing a polymeric composition, the method comprising: a) a determining a first binding energy of an alkoxy or acid substituted monomer to an estradiol related receptor, wherein the alkoxy or acid substituted monomer comprises a bisphenol monomer; b) determining a second binding energy of the corresponding unsubstituted reference monomer to the estradiol related receptor; c) determining the relative binding energy based on the first binding energy and second binding energy; and d) reacting the alkoxy or acid substituted monomer under conditions effective to provide a polymeric composition if the alkoxy or acid substituted monomer exhibits a relative binding energy in the range of from 2.7 kcal/mol to 14 kcal/mol or an intra-molecular hydrogen bond of no greater than 2.1 Å; wherein the polymeric composition does not exhibit a half maximal inhibitory concentration (IC₅₀) less than 0.00025M for a or Pin vitro estradiol receptors.

In still another aspect, the present disclosure provides a method for preparing a polymeric composition, the method comprising: a) providing at least monomer substituted with an oxygenated substituent, wherein the oxygenated substituent comprises at least one alkoxy, hydroxycarbonyl, alkoxycarbonyl, carboxylate, carbonate, aldehyde, or ketone; b) determining the binding energy (BE) of the substituted monomer using a computing device; c) determining the binding energy (BE) of the corresponding unsubstituted reference monomer using a computing device; and d) determining the relative binding energy (RBE) of the substituted monomer to the unsubstituted corresponding reference monomer; and e) reacting the substituted monomer under conditions effective to provide a polymeric composition when the substituted monomer exhibits an RBE in the range of from about 2.7 kcal/mol to about 14 kcal/mol or intra-molecular hydrogen bond is in a range of from 1.7 Å to 2.08 Å.

In yet another aspect, the present disclosure provides a method for preparing a polymeric composition, the method comprising: a) providing at least one alkoxy or acid substituted monomer according to the present disclosure; b) determining the binding energy (BE) of the alkoxy or acid substituted monomer using a computing device; c) determining the binding energy (BE) of the corresponding unsubstituted reference monomer using a computing device; and d) determining the relative binding energy (RBE) of the alkoxy or acid substituted monomer to the unsubstituted corresponding reference monomer; and e) reacting the alkoxy or acid monomer under conditions effective to provide a polymeric composition when the alkoxy or acid monomer exhibits an RBE in the range of from about 2.7 kcal/mol to about 14 kcal/mol or intra-molecular hydrogen bond is in a range of from 1.7 Δ to 2.08 Δ.

In a further aspect, the polymeric compositions comprise monomers that do not exhibit a half maximal inhibitory concentration (IC₅₀) less than 0.00025M for ERR α or ERR β in vitro estradiol receptors.

In one aspect, the polymeric composition is a polycarbonate. In a further aspect, polycarbonates, including polycarbonates of the present disclosure, can be manufactured by processes such as interfacial polymerization and melt polymerization. In a still further aspect, the polycarbonate can, in various aspects, be prepared by a melt polymerization process. Generally, in the melt polymerization process, polycarbonates are prepared by co-reacting, in a molten state, the dihydroxy reactant(s) (i.e., isosorbide, aliphatic diol and/or aliphatic diacid, and any additional dihydroxy compound) and a diaryl carbonate ester, such as diphenyl carbonate, or more specifically, in an aspect, an activated carbonate such as bis(methyl salicyl)carbonate, in the presence of a transesterification catalyst.

While aspects of the present disclosure can be described and claimed in a particular statutory class, such as the system statutory class, this is for convenience only and one of skill in the art will understand that each aspect of the present disclosure can be described and claimed in any statutory class. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon. Nothing herein is to be construed as an admission that the present disclosure is not entitled to antedate such publication by virtue of prior disclosure. Further, the dates of publication provided herein can be different from the actual publication dates, which can require independent confirmation.

### E. ASPECTS

The present disclosure comprises at least the following aspects.

Aspect 1: A method for determining relative binding energy of a monomer, the method comprising: determining a first binding energy of a phenolic monomer, the phenolic monomer having at least one oxygenated substituent in an ortho position of a phenolic group of the phenolic monomer, the oxygenated ortho substituent comprising a structure —OR″, —COOR (carboxylic acid or ester), —O—C(O)R (carboxylate), —O—C(O)—OR (carbonate), or —C(O)—R (aldehyde or ketone), where R is an H, C₁₋₁₈ alkyl, C₆₋₁₈-aryl, or C₇₋₁₈-alkylaryl group and where —OR″ is a C₁₋₁₈ alkyl, C₆₋₁₈-aryl, or C₇₋₁₈-alkylaryl alkoxide; determining a second binding energy of the corresponding unsubstituted reference monomer, wherein the first binding energy and the second binding energy are determined in a ligand binding domain cavity of an estradiol related receptor; and determining the relative binding energy based on the difference between the determined first binding energy and the determined second binding energy.

Aspect 2: The method of aspect 1, wherein the at least one substituent in the ortho position comprises alkoxy, carbonyl, carboxylic acid, carboxylic ester, or carboxylic acid salt, or a combination thereof.

Aspect 3: The method of any of aspects 1-2, wherein the phenolic monomer comprises a bis-phenol monomer.

Aspect 4: The method of any of aspects 1-3, wherein the estradiol related receptor comprises ERR-α or ERR-β.

Aspect 5: The method of any of aspects 1-4, wherein the substituted phenolic monomer comprises at least oxygenated substituent ortho to the phenolic hydroxyl group, the oxygenated ortho substituent comprising an alkoxy or carboxylic acid.

Aspect 6: The method of aspect 5, wherein the intra-molecular hydrogen bond length is the distance between the H of the phenolic hydroxyl group and an oxygen of the alkoxy or carbonyl oxygen of the oxygenated substituent attached in the ortho position of the hydroxyl group.

Aspect 7: The method of aspect 6, wherein the intra-molecular hydrogen bond length is in a range of from about 1.7 to about 2.1 Å.

Aspect 8: The method of any of aspects 1-4, wherein the substituted phenolic monomer is a substituted bisphenol monomer.

Aspect 9: The method of aspect 8, wherein the substituted group is an electronegative group, comprising O, N, or S.

Aspect 10: The method of aspect 9, wherein an intra-molecular hydrogen bond is present between the hydrogen of the phenolic hydroxyl group and the electronegative atom of the substituted group.

Aspect 11: The method of any of aspects 1-10, wherein determining a first binding energy, determining a second binding energy, or determining the relative binding energy is performed using a computing device.

Aspect 12: The method of aspect 11, wherein the computing device comprises a computing system.

Aspect 13: The method of aspect 12, wherein the computing system comprises computing hardware and computing software for performing analysis of data.

Aspect 14: The method of any of aspects 1-13, wherein the binding energy is determined using mathematical analysis techniques.

Aspect 15: The method of any of aspects 1-13, wherein the binding energy is determined using molecular modeling.

Aspect 16: The method of aspect 15, wherein the molecular modeling utilizes quantum mechanics.

Aspect 17: The method of aspect 16, wherein quantum mechanics utilizes density functional theory approach with 6-31G* basis set in conjunction with the B3-LYP exchange-correlation functional.

Aspect 18: The method of any of aspects 1-13, wherein the binding energy value is determined using the formula: binding energy=energy (complex)−[energy(cavity)+energy(monomer)], wherein energy (complex) is the electronic energy of the optimized monomer structure in the constrained structure of the ligand binding domain cavity of the estradiol related receptor, the energy(cavity) is the electronic energy of the cavity and the energy(monomer) is the electronic energy of the monomer.

Aspect 19: The method of aspect 18, wherein the energy of the cavity and the energy of the monomer are calculated in the gas phase with complete optimization of the monomer and unoptimized energy of the protein cavity.

Aspect 20: The method of any of aspects 1-13, wherein the binding energy is determined using constrained geometry optimization.

Aspect 21: The method of any of aspects 1-13, wherein determining the relative binding energy comprises correlating the first and second binding energies.

Aspect 22: The method of any of aspects 1-13, wherein the relative binding energy is determined by comparing the first binding energy with the second binding energy.

Aspect 24: The method of any of aspects 1-13, wherein the relative binding energy is used to determine estradiol activity of previously untested monomers.

Aspect 25: The method of any of aspects 1-13, wherein the relative binding energy permits determination of the estradiol binding activity of untested monomers relative to tested reference monomers.

Aspect 26: The method of any of aspects 1-25, wherein the relative binding energy value is used to determine if the monomer does not exhibit a half maximal inhibitory concentration (IC₅₀) less than 0.00025M for ERR α or ERR β in vitro estradiol receptors.

Aspect 27: The method of any of aspects 1-26, further comprising comparing the relative binding energy to a predefined relative binding energy to determine whether the determined relative binding energy satisfies the predefined relative binding energy; and wherein if the determined relative binding energy satisfies the predefined relative binding energy, the monomer is used to produce a polymeric composition.

Aspect 28: The method of any of aspects 1-27, further comprising reacting the substituted monomer under conditions effective to provide a polymeric composition when the relative binding energy value of the monomer is from about 2.7 kcal/mol to about 14 kcal/mol.

Aspect 29: A method for preparing a polymeric composition, the method comprising: determining a first binding energy of an alkoxy or acid substituted a phenolic monomer in a ligand binding domain cavity of an estradiol related receptor comprising ERR-α or ERR-β, wherein phenolic monomer comprises a bisphenol monomer; determining a second binding energy of the corresponding unsubstituted reference monomer in the same ligand binding domain cavity of an estradiol related receptor comprising ERR-α or ERR-β; determining the relative binding energy based on the first binding energy and second binding energy; and reacting the alkoxy or acid substituted phenolic monomer under conditions effective to provide a polymeric composition if the alkoxy or acid substituted monomer exhibits a relative binding energy in the range of from 2.7 kcal/mol to 14 kcal/mol or an intra-molecular hydrogen bond a range of from about 1.7 Å to about 2.08 Å; wherein the polymeric composition does not exhibit a half maximal inhibitory concentration (IC₅₀) less than 0.00025M for α or β in vitro estradiol receptors.

Aspect 30: The method of aspect 29, wherein the substituted monomer does not exhibit a half maximal inhibitory concentration (IC₅₀) less than 0.00025M for α or β in vitro estradiol receptors.

Aspect 31: The method of aspect 30, wherein the estradiol related receptor comprises ERR-α, ERR-β, or ERR-γ, or a combination thereof.

Aspect 32: The method of aspect 30, wherein the estradiol related receptor is ERR-α.

Aspect 33: The method of aspect 30, wherein the substituted monomer is a bisphenol monomer.

Aspect 34: The method of aspect 30, wherein the polymeric composition is a polycarbonate.

Aspect 35: A method for determining relative binding energy of a monomer, the method comprising: determining a first binding energy (BE) of an alkoxy or acid substituted a phenolic monomer, the phenolic monomer having at least one substituent in an ortho position of a phenolic group of the phenolic monomer; determining a second binding energy (BE) of the corresponding unsubstituted reference monomer, wherein the first binding energy and the second binding energy are determined in a ligand binding domain cavity of an estradiol related receptor comprising ERR-α or ERR-β; and determining the relative binding energy (RBE) based on the first binding energy and second binding energy.

### F. EXAMPLES

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., electronic energies, bond lengths, binding energies, etc.), but some errors and deviations should be accounted for. Unless indicated otherwise, parts are parts by weight, temperature is in ° C. or is at ambient temperature, and pressure is at or near atmospheric. Unless indicated otherwise, percentages referring to composition are in terms of wt %. Unless otherwise indicated, energies are in kcal/mol and bond lengths in Å.

1. General Methods

All materials and reagents were used as is unless otherwise indicated.

Utilizing a conventional in vitro competitive binding assay, estradiol binding activity was quantified by the half maximal inhibitory concentration (IC₅₀) value, which was evaluated for various monomers described herein capable for use as component starting materials in the manufacture of polycarbonate compositions. In various aspects, these starting materials can mimic or replicate various chemical species that could be produced under certain conditions, for example high (pH=8 to 12) or low (pH=1 to 6) pH, as hydrolysis degradation products derived from polymeric materials comprising the component starting materials.

IC₅₀ is defined as the concentration of the test substance at which 50% of the radioligand is displaced from the estradiol related receptor. In a further aspect, IC₅₀ is a quantitative measure that indicates how much of a particular substance, i.e., an inhibitor, is needed to inhibit a given biological process, by one half. IC₅₀ binding concentrations for the alpha in vitro estradiol receptors were tested. Four separate sets of tests were run using a standard competitive binding assay. Samples were dissolved in either ethanol or DMSO. The various compounds were then tested at up to seven different concentrations for each test compound. Each of those tests was run in triplicate. Tests were conducted by displacement of a radio-ligand. For each set of tests a 17P-estradiol control sample was run to ensure proper binding of the natural hormone under the test conditions. Specifically, the IC₅₀ values described herein were determined from in-vitro experiments of the compound binding to ERR-α.

BE is the binding energy (BE) of a particular substance, more specifically a monomer, to the ERR-α receptor, and can be determined using the formula (Eqn 1.) below:

BE=Energy(complex)−[Energy(cavity)+Energy(monomer)].

The BE calculations were performed using the Spartan software from Wavefunction Inc. as described herein. The energy is the electronic energy obtained from the density functional theory (DFT) approach described herein.

Relative binding energy (RBE) is the difference in the calculated binding energies (BE) between a substituted monomer and its corresponding unsubsubstituted monomer as described herein, and can be determined using the formula (Eqn. 2) below:

RBE=BE(substituted monomer)−BE(unsubstituted monomer).

Estradiol and various bisphenol monomers were evaluated to determine IC₅₀. Binding energies (B.E.) in kcal/mol of the monomers described herein were then calculated using the computational methods described herein. The IC₅₀ values from experimental bio-analysis are provided in Table 1.

TABLE 1

| Reference | | | | B.E. |
|---|---|---|---|---|
| ID | Compound | Structure | IC₅₀(nM) | kcal/mol |
| 1 | 17 β Estradiol | 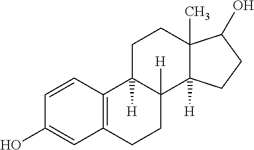 | 10.25 +/− 2.25 | −20.37 |
| 2 | 4,4′-(cyclohexane-1,1- diyl)bis(2-methylphenol) (DMBPC) | 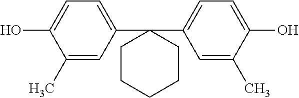 [SMILES: Cc1cc(C2(c3ccc(O)c(C)c3)CCCCC2)ccc1O] | 1275 +/− 789 | −20.17 |
| 3 | 4,4′-(1-phenylethane-1,1- diyl)bis(2-methylphenol) (DMbisAP) | 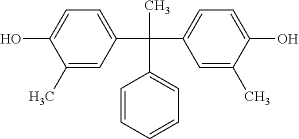 [SMILES: Cc1cc(C(C)(c2ccccc2)c2ccc(O)c(C)c2)ccc1O] | 4800 +/− 1460 | −24.36 |
| 4 | 4,4′-oxydiphenol (DHDE) | 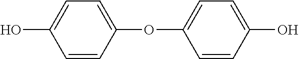 [SMILES: Oc1ccc(Oc2ccc(O)cc2)cc1] | 59670 +/− 46420 | −22.72 |
| 5 | 4,4′-(3,3,5- trimethylcyclohexane-1,1- diyl)diphenol (IPBP) | 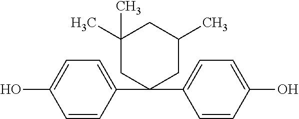 [SMILES: CC1CC(C)(C)CC(c2ccc(O)cc2)(c2ccc(O)cc2)C1] | 455 +/− 133 | −19.61 |
| 6 | 4,4′-(propane-2,2- diyl)bis(2-methylphenol) (DMBPA) | 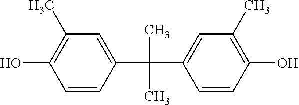 [SMILES: Cc1cc(C(C)(C)c2ccc(O)c(C)c2)ccc1O] | 14000 +/− 3900 | −19.57 |
| 7 | 4,4′-(1,4- phenylenebis(propane-2,2- diyl))diphenol (BPDB) | 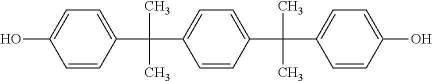 [SMILES: CC(C)(c1ccc(O)cc1)c1ccc(C(C)(C)c2ccc(O)cc2)cc1] | 3920 +/− 840 | −23.50 |
| 8 | bis(4- hydroxyphenyl)methanone (DHBP) | 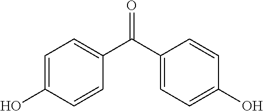 [SMILES: O=C(c1ccc(O)cc1)c1ccc(O)cc1] | 30870 +/− 6795 | −23.97 |
| 9 | 4,4′-(pentane-3,3- diyl)diphenol (BPP) | 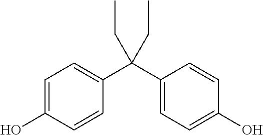 [SMILES: CCC(CC)(c1ccc(O)cc1)c1ccc(O)cc1] | 5.6 +/− 0.4 | −19.31 |
| 10 | Methyl 4,4-bis(4- hydroxyphenyl)pentanoate (MeDPA) | 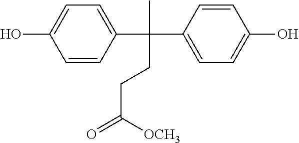 [SMILES: COC(=O)CCC(C)(c1ccc(O)cc1)c1ccc(O)cc1] | 18700 +/− 2410 | −22.85 |
| 11 | (E)-4,4′-(hex-3-ene-3,4- diyl)diphenol (DES) | 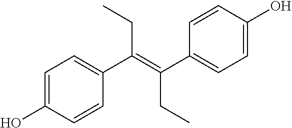 [SMILES: CC/C(=C(/CC)c1ccc(O)cc1)c1ccc(O)cc1] | ** | −23.00 |
| 12 | 4,4′ methylene diphenol (BSF) | 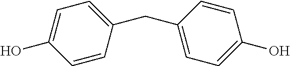 [SMILES: Oc1ccc(Cc2ccc(O)cc2)cc1] | ** | −22.9 |

Various methoxy substituted derivatives of related monomers were also evaluated. Binding energies (B.E.) in kcal/mol, as well as intra-molecular hydrogen bond distance (H—O) between the H of hydroxyl group and the O of —OCH₃ of the monomers described herein were then calculated using the computational methods described herein. Relative BE of the monomers in Table 2 was then calculated with respect to the corresponding unsubstituted reference monomer as described herein.

TABLE 2

| | | | H—-O | B.E. | Relative |
|---|---|---|---|---|---|
| Compound | | | distance | (kcal/ | B.E. |
| (Example ID) | Structure | IC₅₀(nM) | (Å) | Mol) | (kcal/mol) |
| 4,4′-(propane-2,2- diyl)bis(2- methoxyphenol) (PBMP) (Example 1A) | 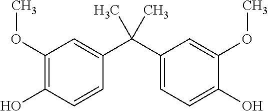 [SMILES: COc1cc(C(C)(C)c2ccc(O)c(OC)c2)ccc1O] | >250000 | 2.08 | −16.93 | 2.94 |
| 4,4′-(cyclohexane-1,1- diyl)bis(2-methoxy-6- methylphenol) (G-DMBPC) (Example 2A) | 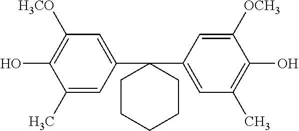 [SMILES: COc1cc(C2(c3cc(C)c(O)c(OC)c3)CCCCC2)cc(C)c1O] | ** | 2.07 | −15.15 | 5.02 |
| 4,4′-(1-phenylethane- 1,1-diyl)bis(2-methoxy- 6-methylphenol) (G-DMbisAP) (Example 3A) | 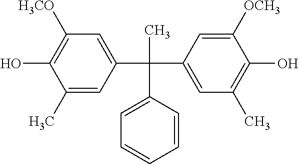 [SMILES: COc1cc(C(C)(c2ccccc2)c2cc(C)c(O)c(OC)c2)cc(C)c1O] | ** | 2.0 | −16.99 | 7.37 |
| 4,4′-oxybis(2- methoxyphenol) G- DHDE (Example 4A) | 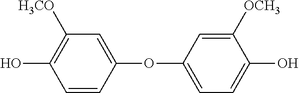 [SMILES: COc1cc(Oc2ccc(O)c(OC)c2)ccc1O] | ** | 2.08 | −17.34 | 5.38 |
| 4,4′-(3,3,5- trimethylcyclohexane- 1,1-diyl)bis(2- methoxyphenol) (G-IPBP) (Example 5A) | 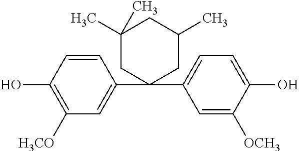 [SMILES: COc1cc(C2(c3ccc(O)c(OC)c3)CC(C)CC(C)(C)C2)ccc1O] | ** | 2.08 | −13.42 | 6.19 |
| 4,4′-(propane-2,2- diyl)bis(2-methoxy-6- methylphenol) (G-DMBPA) (Example 6A) | 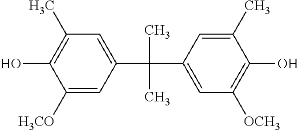 [SMILES: COc1cc(C(C)(C)c2cc(C)c(O)c(OC)c2)cc(C)c1O] | ** | 2.07 | −16.04 | 3.53 |
| 4,4′-(1,4- phenylenebis(propane- 2,2-diyl))bis(2- methoxyphenol) (G-BPDB) (Example 7A) | 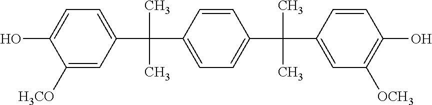 [SMILES: COc1cc(C(C)(C)c2ccc(C(C)(C)c3ccc(O)c(OC)c3)cc2)ccc1O] | ** | 2.08 | −15.75 | 7.75 |
| bis(4-hydroxy-3- methoxyphenyl)methanone (G-DHBP) (Example 8A) | 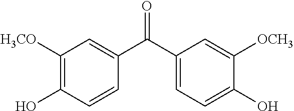 [SMILES: COc1cc(C(=O)c2ccc(O)c(OC)c2)ccc1O] | ** | 2.06 | −16.07 | 7.90 |
| 4,4′-(pentane-3,3- diyl)bis(2- methoxyphenol) (G- BPP) (Example 9A) | 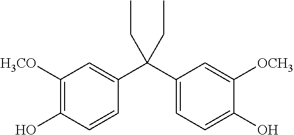 [SMILES: CCC(CC)(c1ccc(O)c(OC)c1)c1ccc(O)c(OC)c1] | ** | 2.081 | −16.59 | 2.72 |
| Methyl 4,4-bis(4- hydroxy-3- methoxyphenyl)pentanoate (G-MeDPA) (Example 10A) | 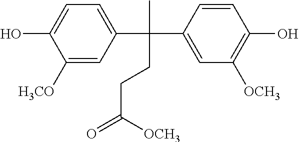 [SMILES: COC(=O)CCC(C)(c1ccc(O)c(OC)c1)c1ccc(O)c(OC)c1] | ** | 2.08 | −16.78 | 6.07 |
| (E)-4,4′-(hex-3-ene-3,4- diyl)bis(2- methoxyphenol) (G- DES) (Example 11A) | 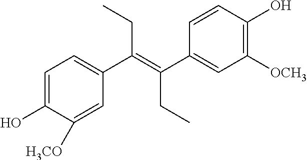 [SMILES: CC/C(=C(/CC)c1ccc(O)c(OC)c1)c1ccc(O)c(OC)c1] | ** | 2.07 | −14.76 | 8.24 |
| 5,5′ Methylene bis (2- hydroxy benzoic acid) (MdSA) (Example 12A) | 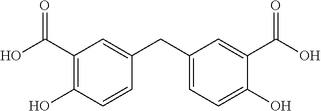 [SMILES: O=C(O)c1cc(Cc2ccc(O)c(C(=O)O)c2)ccc1O] | >250000 | 1.74 | −9.109 | 13.79 |

In one aspect, the negative BE values indicate that the binding of monomers to the LBD cavity of ERR-α is exothermic with respect to the separated elements (i.e. cavity and ligand) as described by Eqn 1. As shown in FIG. 2, the phenolic OH groups of 17-β estradiol form an inter-molecular hydrogen bond of 1.7 Å with the carbonyl group of Glu and an inter-molecular hydrogen bond with the water and Arg at 2.04 and 2.34 Å, respectively. The alcoholic OH forms an inter-molecular hydrogen bond with the N of His at 1.9 Å.

As such, the monomers presented in Table 1 possess only intermolecular hydrogen bond with the amino acids of the ligand binding domain cavity, and do not possess intra-molecular hydrogen bonds as described in various aspects of the present disclosure.

As briefly described, the binding energies presented in Table 2 are for the ortho methoxy (—OCH₃) substituted derivatives of monomers presented in Table 1 (e.g., example 1A, 2A, 3A . . . 12A represent the ortho methoxy (—OCH₃) substituted derivatives of unsubstituted reference monomers identified as 1, 2, 3 . . . 12, in Table 1, respectively). The O—H distances presented in Table 2 are the intra-molecular hydrogen bond between the H of the hydroxyl group and the O of the —OCH₃ group. The intra-molecular hydrogen bond of the monomers described in Table 2 corresponds to around 2.07-2.08 Å. In various aspects, in order to be able to bind to the ligand binding domain (LBD) cavity of the ERR-α through inter-molecular hydrogen bonds, the intra-molecular hydrogen bonds of these monomers need to be weakened or broken. It is believed that monomers possessing strong intra-molecular hydrogen bonds, such as the monomers described in the present disclosure, will compete with the intermolecular hydrogen bonding to the LBD cavity and will reduce the binding energy of the monomer towards the LBD cavity, and reduce the binding affinity of the monomers. As the intra-molecular hydrogen bond weakens by increasing the intra-molecular bond length toward 3.0 Å or beyond, the binding of monomers to the LBD cavity may also increase.

The relative binding energy (RBE) presented in Table 2 is the difference in the binding energy (BE) between the methoxy derivative monomer and its corresponding unsubstituted reference monomer. The positive values of RBE in Table 2 indicate that the binding energy of the methoxy substituted monomers will have reduced binding affinity towards ERR α. As shown in Table 2, all methoxy substituted monomers resulted in an increase in relative binding energy (RBE) with respect to their corresponding bisphenol monomer, having a RBE in the range of from 2.72 kcal/mol for G-BPP to 8.24 kcal/mol for G-DES. The increase in RBE values also indicate that the BE of the substituted monomer is less exothermic with respect to the unsubstituted monomer. This indicates a reduced binding activity of substituted monomer with respect to the unsubstituted monomer to the LBD cavity. This data suggest that the presence of strong intra-molecular hydrogen bonding will increase the relative binding energy of the monomers and as a result reduce the binding affinity to the ligand binding domain (LBD) of the ERR-α. The RBE corresponding to zero or negative values will indicate the same or a higher binding affinity. Without wishing to be bound by a particular theory, the increase in RBE is due to the strong intra-molecular hydrogen bonding observed in methoxy substituted bisphenols. Interestingly, the calculated RBE value for PBMP supports the conclusion that methoxy substituted monomers having a reduced RBE, such as the PBMP monomer, exhibit reduced or no detectable estradiol binding activity, for example, with the LBD cavity of ERR-α.

Additionally, substitution of an acid group in the ortho position of a bisphenol also yielded an increase in relative binding energy. As seen with BSF, the acid substituted MdSA monomer has a RBE of 13.79 kcal/mol with respect to the unsubstituted reference monomer, BSF. Moreover, as shown in FIG. 3B, the intra-molecular hydrogen bond distance between the H of hydroxyl group and the carbonyl O of —COOH as shown in FIG. 3B forms an intra-molecular hydrogen bond corresponding to 1.74 Å.

While calculated BE values may vary depending upon the parameters, the model of the protein structure, and the level of theory used, the results suggest the decrease in the binding affinity towards estradiol receptors is due to the strong intra-molecular hydrogen bonding and increased relative binding energy (as compared with their corresponding monomers without intra-molecular hydrogen bonds).

In further aspects, at least one step of the disclosed methods is performed by a computing device. In one aspect, the computing device can comprise a computing system. In a further system, the computing system generally comprises computing hardware and computing software for performing various computing tasks or instructions, for example, molecular modeling and analysis of data. In a still further aspect, the computing software can comprise any desired molecular modeling program. Those of skill in the art will recognize that the computing system can be configured in a number of ways using known computing hardware and known computing software, such as, for example, Spartan software from Wavefunction Inc. as described herein.

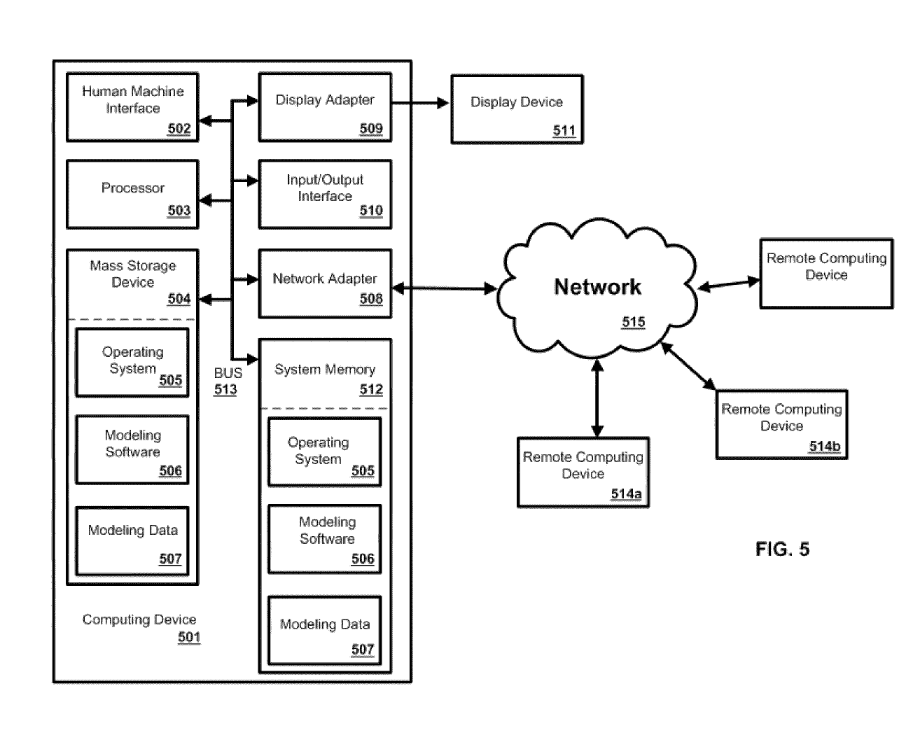

In an exemplary aspect, the methods can be implemented on a computing system such as computing device as illustrated in FIG. 5 and described below. Similarly, the methods disclosed can utilize one or more computers to perform one or more functions in one or more locations. FIG. 5 is a block diagram illustrating an exemplary operating environment for performing the disclosed methods. This exemplary operating environment is only an example of an operating environment and is not intended to suggest any limitation as to the scope of use or functionality of operating environment architecture. Neither should the operating environment be interpreted as having any dependency or requirement relating to any one or combination of components illustrated in the exemplary operating environment.

The present methods and systems can be operational with numerous other general purpose or special purpose computing system environments or configurations. Examples of well known computing systems, environments, and/or configurations that can be suitable for use with the systems and methods comprise, but are not limited to, personal computers, server computers, laptop devices, and multiprocessor systems. Additional examples comprise set top boxes, programmable consumer electronics, network PCs, minicomputers, mainframe computers, distributed computing environments that comprise any of the above systems or devices, and the like.

The processing of the disclosed methods and systems can be performed by software components. The disclosed systems and methods can be described in the general context of computer-executable instructions, such as program modules, being executed by one or more computers or other devices. Generally, program modules comprise computer code, routines, programs, objects, components, data structures, etc. that perform particular tasks or implement particular abstract data types. The disclosed methods can also be practiced in grid-based and distributed computing environments where tasks are performed by remote processing devices that are linked through a communications network. In a distributed computing environment, program modules can be located in both local and remote computer storage media including memory storage devices.

The components of the computing device 501 can comprise, but are not limited to, one or more processors 503, a system memory 512, and a system bus 513 that couples various system components including the processor 503 to the system memory 512. In the case of multiple processors 503, the system can utilize parallel computing.

The system bus 513 represents one or more of several possible types of bus structures, including a memory bus or memory controller, a peripheral bus, an accelerated graphics port, and a processor or local bus using any of a variety of bus architectures. By way of example, such architectures can comprise an Industry Standard Architecture (ISA) bus, a Micro Channel Architecture (MCA) bus, an Enhanced ISA (EISA) bus, a Video Electronics Standards Association (VESA) local bus, an Accelerated Graphics Port (AGP) bus, and a Peripheral Component Interconnects (PCI), a PCI-Express bus, a Personal Computer Memory Card Industry Association (PCMCIA), Universal Serial Bus (USB) and the like. The bus 513, and all buses specified in this description can also be implemented over a wired or wireless network connection and each of the subsystems, including the processor 503, a mass storage device 504, an operating system 505, network software 506, network data 507, a network adapter 508, system memory 512, an Input/Output Interface 510, a display adapter 509, a display device 511, and a human machine interface 502, can be contained within one or more remote computing devices 514a,b,c at physically separate locations, connected through buses of this form, in effect implementing a fully distributed system.

The computing device 501 typically comprises a variety of computer readable media. Exemplary readable media can be any available media that is accessible by the computing device 501 and comprises, for example and not meant to be limiting, both volatile and non-volatile media, removable and non-removable media. The system memory 512 comprises computer readable media in the form of volatile memory, such as random access memory (RAM), and/or non-volatile memory, such as read only memory (ROM). The system memory 512 typically contains data such as network data 507 and/or program modules such as operating system 505 and network software 506 that are immediately accessible to and/or are presently operated on by the processor 503.

In another aspect, the computing device 501 can also comprise other removable/non-removable, volatile/non-volatile computer storage media. By way of example, FIG. 5 illustrates a mass storage device 504 which can provide non-volatile storage of computer code, computer readable instructions, data structures, program modules, and other data for the computing device 501. For example and not meant to be limiting, a mass storage device 504 can be a hard disk, a removable magnetic disk, a removable optical disk, magnetic cassettes or other magnetic storage devices, flash memory cards, CD-ROM, digital versatile disks (DVD) or other optical storage, random access memories (RAM), read only memories (ROM), electrically erasable programmable read-only memory (EEPROM), and the like.

Optionally, any number of program modules can be stored on the mass storage device 504, including by way of example, an operating system 505 and modeling software 506. Each of the operating system 505 and modeling software 506 (or some combination thereof) can comprise elements of the programming and the modeling software 506. Modeling data 507 can also be stored on the mass storage device 504. Modeling data 507 can be stored in any of one or more databases known in the art. Examples of such databases comprise, DB2®, Microsoft® Access, Microsoft® SQL Server, Oracle®, mySQL, PostgreSQL, and the like. The databases can be centralized or distributed across multiple systems.

In another aspect, the user can enter commands and information into the computing device 501 via an input device (not shown). Examples of such input devices comprise, but are not limited to, a keyboard, pointing device (e.g., a “mouse”), a microphone, a joystick, a scanner, tactile input devices such as gloves, and other body coverings, and the like. These and other input devices can be connected to the processor 503 via a human machine interface 502 that is coupled to the system bus 513, but can be connected by other interface and bus structures, such as a parallel port, game port, an IEEE 1394 Port (also known as a Firewire port), a serial port, or a universal serial bus (USB).

In yet another aspect, a display device 511 can also be connected to the system bus 513 via an interface, such as a display adapter 509. It is contemplated that the computing device 501 can have more than one display adapter 509 and the computing device 501 can have more than one display device 511. For example, a display device can be a monitor, an LCD (Liquid Crystal Display), or a projector. In addition to the display device 511, other output peripheral devices can comprise components such as speakers (not shown) and a printer (not shown) which can be connected to the computing device 501 via Input/Output Interface 510. Any step and/or result of the methods can be output in any form to an output device. Such output can be any form of visual representation, including, but not limited to, textual, graphical, animation, audio, tactile, and the like. The display 511 and computing device 501 can be part of one device, or separate devices.

The computing device 501 can operate in a networked environment using logical connections to one or more remote computing devices 514a,b,c. By way of example, a remote computing device can be a personal computer, portable computer, a smart phone, a server, a router, a network computer, a peer device or other common network node, and so on. Logical connections between the computing device 501 and a remote computing device 514a,b,c can be made via a network 515, such as a local area network (LAN) and a general wide area network (WAN). Such network connections can be through a network adapter 508. A network adapter 508 can be implemented in both wired and wireless environments. Such networking environments are conventional and commonplace in dwellings, offices, enterprise-wide computer networks, intranets, and the Internet.

For purposes of illustration, application programs and other executable program components such as the operating system 505 are illustrated herein as discrete blocks, although it is recognized that such programs and components reside at various times in different storage components of the computing device 501, and are executed by the data processor(s) of the computer. An implementation of modeling software 506 can be stored on or transmitted across some form of computer readable media. Any of the disclosed methods can be performed by computer readable instructions embodied on computer readable media. Computer readable media can be any available media that can be accessed by a computer. By way of example and not meant to be limiting, computer readable media can comprise “computer storage media” and “communications media.” “Computer storage media” comprise volatile and non-volatile, removable and non-removable media implemented in any methods or technology for storage of information such as computer readable instructions, data structures, program modules, or other data. Exemplary computer storage media comprises, but is not limited to, RAM, ROM, EEPROM, flash memory or other memory technology, CD-ROM, digital versatile disks (DVD) or other optical storage, magnetic cassettes, magnetic tape, magnetic disk storage or other magnetic storage devices, or any other medium which can be used to store the desired information and which can be accessed by a computer.

The methods and systems can employ Artificial Intelligence techniques such as machine learning and iterative learning. Examples of such techniques include, but are not limited to, expert systems, case based reasoning, Bayesian networks, behavior based AI, neural networks, fuzzy systems, evolutionary computation (e.g. genetic algorithms), swarm intelligence (e.g. ant algorithms), and hybrid intelligent systems (e.g. expert inference rules generated through a neural network or production rules from statistical learning).

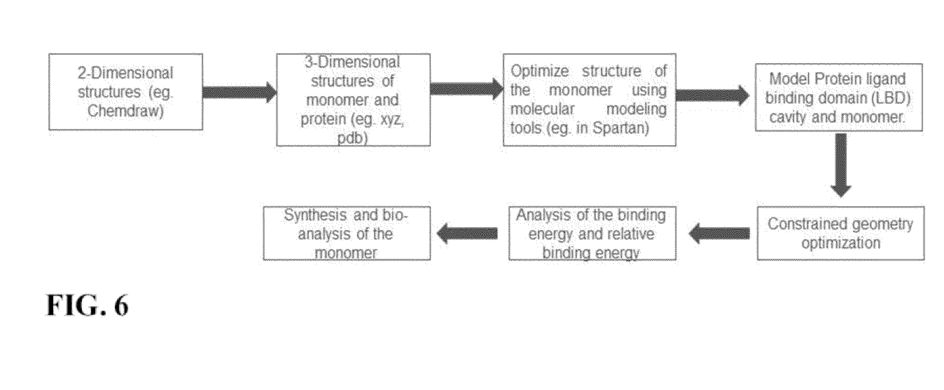

FIG. 6 shows an exemplary schematic flow chart of certain aspects of the disclosure that relates to the methods that may be supported by the hardware and software described elsewhere herein. For example, in certain aspects, the method comprises (a) developing ligand structural data comprising monomer structural data of two-dimensional drawings showing molecular connectivity; (b) converting these two-dimensional drawings to three-dimensional format (e.g. *.mol, *.xyz, *.spartan) using available computing software known to those of skill in the art, or deriving three-dimensional structural data from experimentally-determined data; (c) optimizing the three dimensional structure of the molecule using any of a variety of available quantum mechanical methods; (d) modeling the interaction between the protein ligand binding domain cavity and the monomer; (e) determining the binding energy between monomer and the cavity using a constrained geometry optimization; (f) comparing the relative binding energies of the substituted and unsubstituted reference monomers; and (g) synthesizing and analyzing the resulting monomer and monomer product.

It will be apparent to those skilled in the art that various modifications and variations can be made in the present disclosure without departing from the scope or spirit of the disclosure. Other aspects of the disclosure will be apparent to those skilled in the art from consideration of the specification and practice of the disclosure disclosed herein. It is intended that the specification and examples be considered as exemplary only, with a true scope and spirit of the disclosure being indicated by the following claims.

The patentable scope of the disclosure is defined by the claims, and can include other examples that occur to those skilled in the art. Such other examples are intended to be within the scope of the claims if they have structural elements that do not differ from the literal language of the claims, or if they include equivalent structural elements with insubstantial differences from the literal languages of the claims.

What is claimed is:

1. A method for determining relative binding energy of a monomer, the method comprising:
a) determining a first binding energy of a phenolic monomer, the phenolic monomer having at least one oxygenated substituent in an ortho position of a phenolic group of the phenolic monomer, the oxygenated ortho substituent comprising a structure —OR″, —COOR (carboxylic acid or ester), —O—C(O)R (carboxylate), —O—C(O)—OR (carbonate), or —C(O)—R (aldehyde or ketone), where R is an H, C₁₋₁₈ alkyl, C₆₋₁₈-aryl, or C₇₋₁₈-alkylaryl group and where —OR″ is a C₁₋₁₈ alkyl, C₆₋₁₈-aryl, or C₇₋₁₈-alkylaryl alkoxide;
b) determining a second binding energy of the corresponding unsubstituted reference monomer, wherein the first binding energy and the second binding energy are determined in a ligand binding domain cavity of an estradiol related receptor; and
c) determining the relative binding energy based on the difference between the determined first binding energy and the determined second binding energy.

2. The method of claim 1, wherein the at least one substituent in the ortho position comprises alkoxy, carbonyl, carboxylic acid, carboxylic ester, or carboxylic acid salt, or a combination thereof.

3. The method of claim 1, wherein the phenolic monomer comprises a bis-phenol monomer.

4. The method of claim 1, wherein the estradiol related receptor comprises ERR-α or ERR-β.

5. The method of claim 1, wherein the substituted phenolic monomer comprises at least oxygenated substituent ortho to the phenolic hydroxyl group, the oxygenated ortho substituent comprising an alkoxy or carboxylic acid.

6. The method of claim 5, wherein an intra-molecular hydrogen bond length is in a range of from about 1.7 to about 2.1 Å, and wherein the intra-molecular hydrogen bond length is the distance between the H of the phenolic hydroxyl group and an oxygen of the alkoxy or carbonyl oxygen of the oxygenated substituent attached in the ortho position of the hydroxyl group.

7. The method of claim 1, wherein the substituted phenolic monomer is a substituted bisphenol monomer.

8. The method of claim 7, wherein the substituted group is an electronegative group, comprising O, N, or S.

9. The method of claim 8, wherein an intra-molecular hydrogen bond is present between the hydrogen of the phenolic hydroxyl group and the electronegative atom of the substituted group.

10. The method of claim 1, wherein determining a first binding energy, determining a second binding energy, or determining the relative binding energy is performed using a computing device.

11. The method of claim 1, wherein the binding energy is determined using mathematical analysis techniques.

12. The method of claim 1, wherein the binding energy is determined using molecular modeling.

13. The method of claim 12, wherein the molecular modeling utilizes quantum mechanics.

14. The method of claim 13, wherein quantum mechanics utilizes density functional theory approach with 6-31G* basis set in conjunction with the B3-LYP exchange-correlation functional.

15. The method of claim 1, wherein the binding energy value is determined using the formula: binding energy=energy (complex)−[energy(cavity)+energy(monomer)], wherein energy (complex) is the electronic energy of the optimized monomer structure in the constrained structure of the ligand binding domain cavity of the estradiol related receptor, the energy(cavity) is the electronic energy of the cavity and the energy(monomer) is the electronic energy of the monomer.

16. The method of claim 15, wherein the energy of the cavity and the energy of the monomer are calculated in the gas phase with complete optimization of the monomer and unoptimized energy of the protein cavity.

17. The method of claim 1, wherein one or more of the first binding energy and the second binding energy is determined using constrained geometry optimization.

18. The method of claim 1, wherein determining the relative binding energy comprises correlating the first and second binding energies.

19. The method of claim 1, wherein the relative binding energy is determined by comparing the first binding energy with the second binding energy.

20. The method of claim 1, wherein the relative binding energy is used to determine estradiol activity of previously untested monomers.

21. The method of claim 1, wherein the relative binding energy permits determination of the estradiol binding activity of untested monomers relative to tested reference monomers.

22. The method of claim 1, wherein the relative binding energy value is used to determine if the monomer does not exhibit a half maximal inhibitory concentration (IC₅₀) less than 0.00025M for ERR α or ERR β in vitro estradiol receptors.

23. The method of claim 1, further comprising comparing the relative binding energy to a predefined relative binding energy to determine whether the determined relative binding energy satisfies the predefined relative binding energy; and wherein if the determined relative binding energy satisfies the predefined relative binding energy, the monomer is used to produce a polymeric composition.

24. The method of claim 1, further comprising reacting the substituted monomer under conditions effective to provide a polymeric composition when the relative binding energy value of the monomer is from about 2.7 kcal/mol to about 14 kcal/mol.

25. A method for preparing a polymeric composition, the method comprising:
a) determining a first binding energy of an alkoxy or acid-substituted a phenolic monomer in a ligand binding domain cavity of an estradiol related receptor comprising ERR-α or ERR-β, wherein phenolic monomer comprises a bisphenol monomer;
b) determining a second binding energy of the corresponding unsubstituted reference monomer in the same ligand binding domain cavity of an estradiol related receptor comprising ERR-α or ERR-β;
c) determining the relative binding energy based on the first binding energy and second binding energy; and
d) reacting the alkoxy or acid substituted phenolic monomer under conditions effective to provide a polymeric composition if the alkoxy or acid substituted monomer exhibits a relative binding energy in the range of from 2.7 kcal/mol to 14 kcal/mol or an intra-molecular hydrogen bond a range of from about 1.7 Å to about 2.08 Å;
wherein the polymeric composition does not exhibit a half maximal inhibitory concentration (IC₅₀) less than 0.00025M for α or β in vitro estradiol receptors.

26. The method of claim 25, wherein the substituted monomer does not exhibit a half maximal inhibitory concentration (IC₅₀) less than 0.00025M for alpha or beta in vitro estradiol receptors.

27. The method of claim 25, wherein the estradiol related receptor comprises ERR-α, ERR-β, or ERR-γ, or a combination thereof.

28. The method of claim 25, wherein the estradiol related receptor is ERR-α.

29. The method of claim 25, wherein the substituted monomer is a bisphenol monomer.

30. The method of claim 25, wherein the polymeric composition is a polycarbonate.
